(19) United States
(12) Patent Application Publication
(10) Pub. No.: US 2006/0167494 A1
(43) Pub. Date: Jul. 27, 2006

(54) Aneurysm repair method and apparatus

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/337,075

(22) Filed: Jan. 23, 2006

Related U.S. Application Data
(60) Provisional application No. 60/645,043, filed on Jan. 21, 2005.

(51) Int. Cl. A61B 17/08

(52) U.S. Cl. 606/213

(57) ABSTRACT

Sheaths of fabric material are compressed on either side of a separator by manipulating internal and external catheters to form disks which sandwich the neck of an aneurysm to obliterate the aneurysm.

This application claims benefit under 35 USC 119(a) from provisional patent application 60/645043, filed Jan. 21, 2005.

### BACKGROUND OF THE INVENTION

Aneurysmal subarachnoid hemorrhage affects 10 of every 100,00 people per year in the United States. Any individual experiencing such an event faces some sobering statistics. One third will die, one third will suffer some permanent neurological or cognitive disability and only one third will survive to continue some modicum of a normal life.

Aneurysmal subarachnoid hemorrhage occurs when a weakened area on a cerebral blood vessel bursts allowing blood to escape and damage surrounding delicate brain structures.

The blood that escapes the confines of a blood vessel can cause further deleterious effects such as compressive hematomas, hydrocephalus or vasospasm.

The time honored approach to repairing the weakened area is surgical. Because aneurysms are generally a saccular or berry shaped dilatation of an artery wall, traditional surgical approaches have focused on ligation or clipping of the neck of the lesion and thereby obliterating or excluding it from the cerebrovascular circulation. Such approaches involve a direct attack on the aneurysm which requires a craniotomy or an operative opening of the skull. These procedures are major operations which, in and of themselves, carry significant risk.

In an effort to avoid a craniotomy to exclude aneurysms from the cerebrovascular circulation newer techniques have focused on endovascular approaches. Endovascular approaches fall within the realm of minimally invasive surgery and generally entail the placement of a balloon or special metallic coils within the aneurysm cavity to obliterate it or cause thrombosis of the aneurysmal sac. Additionally, endovascular stents are employed either alone or in conjunction with other techniques in an effort to exclude aneurysms from the cerebrovascular circulation. These internal repair processes involve the use of special catheters that gain access to the circulation via a vessel in the arm or leg and are guided to the aneurysmal site by fluoroscopic x-ray control.

The problem with balloon occlusion of an aneurysm sac is that inflation of any object within the lesion can cause aneurysmal rupture with devastating consequences. The problem with inserting detachable coils into the aneurysmal sac to cause thrombosis is that this also includes the risk of aneurysmal rupture, but additionally includes problems of incomplete thrombosis or extension of the thrombosis into the parent artery with subsequent distal embolism and stroke. In addition, even in situations where aneurysmal coiling appears adequate and complete thrombosis of the sac occurs, delayed recanalization of the aneurysmal sac can happen with the risk or recurrent hemorrhage being an ever present threat.

The problem with endovascular stents is that most aneurysm necks are situated at the bifurcation of major blood vessels making stent obliteration of aneurysms difficult or impossible without compromising the flow of blood past the aneurysmal neck and into the important adjacent bifurcations. Clearly, the art of endovascular neurosurgery requires a better way to effectively treat these lesions and permanently obliterate them as a pathologic entity.

### SUMMARY OF THE INVENTION

An object of this invention is to provide for a better way of repairing aneurysms endovascularly without the drawbacks of using balloons or coils.

Another objective of this invention to focus on direct repair of the aneurysmal neck, rather than relying on filling the delicate and fragile sac fundus with balloon material, coils, or unreliable thrombus.

A further objective of the invention is to provide a technically easier way of repairing aneurysms while simultaneously utilizing familiar catheter-based endovascular techniques.

To achieve these objectives, a dual catheter or catheter within a catheter device is employed. In this design, the inner catheter can be slid within the outer catheter which acts as a sleeve. The tip of the inner catheter protrudes beyond the confines of the outer catheter to form the working tip. The catheters support compressible fabric sheaths and deform them to repair an aneurysm, as described below.

### BRIEF DESCRIPTION OF THE DRAWINGS

In the accompanying drawings:

FIG. 1 is an elevational view of an aneurysm sealing tool embodying the invention;

FIG. 2 shows the tool partly inserted from a blood vessel into an aneurysm;

FIGS. 3-5 show the compression sheaths being progressively expanded to seal of the aneurysm, and

FIG. 6 shows the sheaths fully expanded, and severed from the installation catheters.

### DESCRIPTION OF THE PREFERRED EMBODIMENTS

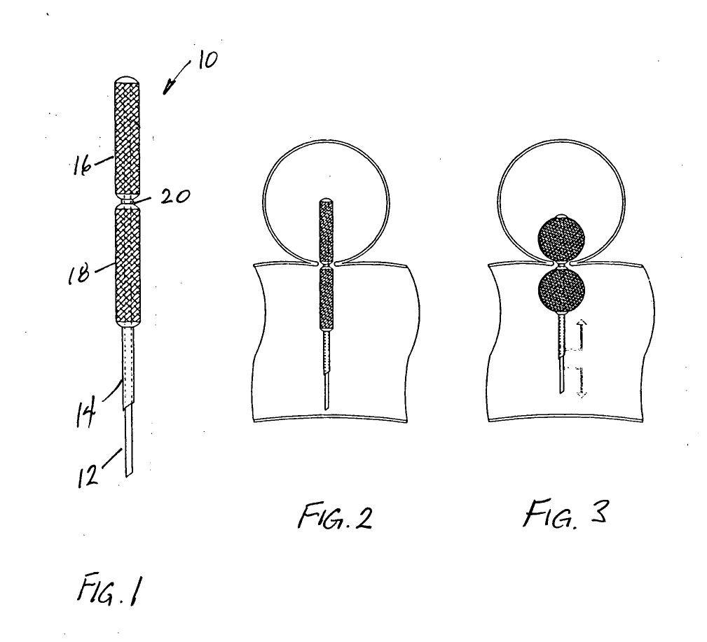

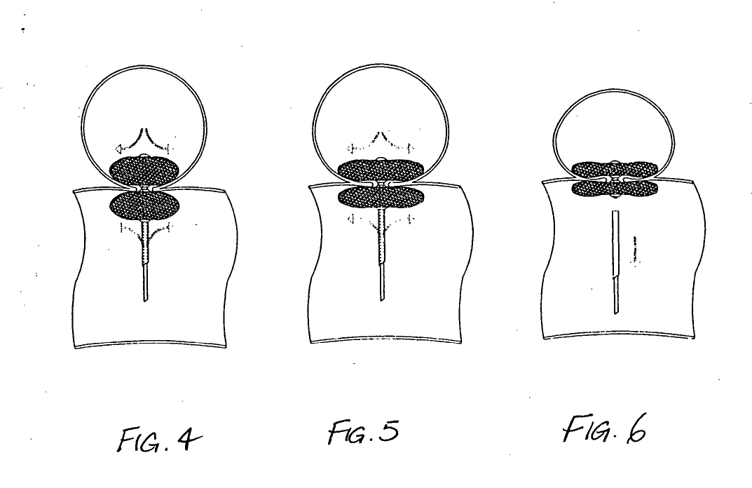

As shown in FIG. 1, a device 10 embodying the invention is placed at the distal end of relatively movable inner and outer catheters 12,14. To the working tip of the inner catheter 12 at least one sheath 16 of a fabric material having sufficient elasticity and tensile strength to be utilized in vessel wall repair. Any fabric presently employed in vascular surgery such as Dacron, Gortex, or polypropylene could be used. For the present invention, the fabric chosen for use should be both biocompatible and have elastomeric properties sufficient to allow it to return to its predeployment shape when not mechanically stressed.

The sheath 16 of fabric is, in the preferred embodiments, attached to the distal end of the inner catheter 12 at the working tip. The distal end of the fabric sheath is secured to the distal end of the inner catheter by an O-ring (not shown) or other means of intimate bonding. The proximal end of the sheath is not attached to the inner catheter, so that when the outer catheter is slid over the inner catheter toward the tip, it compresses the fabric sheath longitudinally, causing the sheath to balloon outward in a fusiform fashion. As the outer sheath advances further toward the O-ring or distal tip of the inner catheter, the fabric sheath shortens further to form an oblate spheroid shape and then a discoid shape. As the longitudinal compression of the fabric sheath continues, its proximal and distal ends eventually meet, leaving the fabric in a flattened disc-like shape which has a diameter that approximates the length of the sheath prior to the initiation of longitudinal compression. It is the final disc-like shape of the fabric sheath that occludes the opening of the aneurysmal neck.

By using two sheaths 16,18 in series (one on either side of the separator 20), as shown in the drawings, and compressing them simultaneously as described above, the opening of the neck of the aneurysm can be effectively obliterated by sandwiching the stoma of the aneurysm between two discs of fabric which are then fixed in position, detached and left in situ. Alternatively, a single discoid sheath could be used in conjunction with a stent to allow effective obliteration of a suitable aneurysm.

Another alternative is that the fabric used for repair of the aneurismal stoma may be one having shape memory (e.g., electrometric) properties, and constructed to have a discoid shape when at rest. In this case, the outer catheter is connected to the proximal end of the fabric, and the discoid shape is then stretched into a sheath-like shape over the inner catheter by applying and maintaining traction to the outer catheter. Once the fabric sheath or sheaths have been positioned appropriately across the stoma of an aneurysm, the outer sheath traction is relaxed, allowing the fabric to spontaneously return to its original discoid shape by virtue of its shape memory properties.

In either embodiment, the aneurismal stoma is obliterated by a discoid patch or patches of fabric. In the first embodiment, this is achieved by compression of the fabric sheath(s) with the outer catheter, whereas in the second embodiment, it is achieved by relaxing longitudinal tension on the fabric sheath which then spontaneously assumes a planar discoid shape by virtue of its shape memory properties and its tendency to return to its initial discoid shape.

I claim:

1. A device for endovascular repair of vascular aneurysms or other defects in vessel walls, said device comprising at least one deployable fabric sheath which can change shape when longitudinally compressed, and means for longitudinally compressing the sheath.

2. The invention of claim 1, wherein the compressing means comprise inner and outer catheters, the inner catheter being retractable with respect to the outer catheter, and wherein the fabric sheath encircles the inner catheter.

3. The invention of claim 1, wherein the distal end of the fabric sheath is attached to a distal component of the catheter.

4. The invention of claim 1, wherein the fabric sheath or sheaths are detachable from the compressing means once suitably deployed.

5. The invention of claim 1, wherein the fabric sheath is a synthetic polymer fabric.

6. The invention of claim 5, wherein the synthetic polymer is polypropylene.

7. The invention of claim 5, wherein the synthetic polymer is polyester.

8. The invention of claim 1, wherein the fabric sheath is a natural or synthetic fabric whose shape can be altered from a cylindrical or sheath-like shape to a globular or discoid shape when compressed lengthwise.

9. A device for endovascular repair of vascular aneurysms or other defects in vessel walls, said device comprising a deployable fabric sheath having shape memory properties and constructed to form a globular or discoid shape when longitudinal traction on it is relaxed.

10. The invention of claim 9, wherein the fabric sheath has electrometric properties.

11. The invention of claim 9, wherein the fabric sheath is a synthetic polymer.

12. The invention of claim 11, wherein the synthetic polymer is a polypropylene.

13. The invention of claim 9, wherein the fabric sheath is only natural or synthetic fabric which has sufficient shape memory properties that it will change from a sheath-like or cylindrical shape to a globular or discoid shape when longitudinal traction on it is relaxed.

14. A device for endovascular repair of vascular aneurysms or other defects in vessel wall, which repairs the defect by trapping it between layers of pliable synthetic material.
